(19) United States
(12) Patent Application Publication
(10) Pub. No.: US 2007/0202560 A1
(43) Pub. Date: Aug. 30, 2007

(54) Resin microchannel array, method of manufacturing the same and blood test method using the same

(75) Inventors: Yuji Kikuchi, Tokyo (JP); Taiji Nishi, Tokyo (JP); Motohiro Fukuda, Tsukuba-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: National Food Research Institute, Tsukuba-shi (JP); Kuraray Co., Ltd., Kurashiki-shi (JP)

(21) Appl. No.: 11/360,672

(22) Filed: Feb. 24, 2006

(51) Int. Cl. C12Q 1/54; C12M 3/00

(52) U.S. Cl. 435/14; 435/287.1

(57) ABSTRACT

A resin microchannel array includes a first substrate having a plurality of depressions, each depression having an inlet port at one end and an outlet port at another end, and walls sectioning the depressions, each wall having a micro groove connecting the depressions, and a second substrate having a flat surface bonded or pressure-contacted to a surface of the first substrate. Spaces created by the depressions and the grooves in a bonded or pressure contacted part between the first substrate and the second substrate serve as flow channels. Each of a width and a depth of the flow channel is within a range of 1 to 50 μm, and a ratio of the width and the depth of the flow channel is within a range of 1:10 to 10:1.

### BACKGROUND OF THE INVENTION

1. Field of the Invention

The present invention relates to a resin microchannel array suitable for health care and diagnosis and treatment of disease, a method of manufacturing the resin microchannel array, and a method of testing blood.

2. Description of the Related Art

As societies mature, values on medical care and health have changed. People now seek healthy and high-quality life, not merely primary health care. It is expected that more and more individuals will place a higher value on preventive medicine than on curative medicine because of increase in medical care costs, disease prevention being less costly than treatment, and increase in the number of those who are in between healthy and diseased.

On this account, in the medical field, and particularly in the clinical laboratory field, there is an increasing need for a non-restraint examination system that enables prompt examination and diagnosis in the vicinity of a patient such as at an operating room, bedside and home, and a noninvasive or minimally invasive examination system that requires only a small amount of sample of blood and so on.

The measurement and evaluation of formed elements of blood, which is red blood cells, white blood cells and blood platelets, are essential for health care and diagnosis and treatment of disease. In order to measure the red blood cell deformability, the ability of blood to pass through a film having minute openings such as Nuclepore filter and nickel mesh filter has been examined. For the measurement of platelet aggregability, a method of measuring a change in the turbidity of platelet suspension that accompanies the platelet aggregation has been used. Further, for the measurement of white blood cell activity, Boyden chamber method, particle phagocytosis test, chemiluminescence method and so on have been used according to several aspects of the white blood cell activity. The white blood cell activity is particularly important for infection, immunotherapy, immunosuppressive therapy and so on.

However, the above measurement methods have problems such as low efficiency, low reproducibility and low quantitative ability, and therefore they fail to serve as effective measurement methods that are adequate for the importance of measurement. Further, the conventional platelet aggregability measurement method requires time and labor for sample preparation and its sensitivity is not sufficient.

Furthermore, the conventional red blood cell deformability measurement method is lack of reliability since openings or grooves can be obstructed by the formed elements in blood sample during measurement.

In addition, a method that separates a single kind of blood cell fraction from blood sample and measure it for the purpose of preventing interference with other kinds of blood cells. However, this method not only requires much time and labor but also fails to avoid the denaturation of blood cells during the preparation or the denaturation due to the separation process. The physiological or diagnostic values of the measurement results are thereby low.

Besides, though the measurement by completely separating the passive movement of blood cells due to hydrostatic pressure different and the active movement of blood cells due to biologically active substance stimulation, and the effects of mechanical stress on blood cells are meaningful for research and diagnosis, there has been no way to enable quantitative research on these issues presently.

To eliminate the above problems, it has been proposed to manufacture a microchannel array with semiconductor microfabrication technology, which patterns a silicon substrate by photolithography and micro-fabricates flow channels on the silicon substrate by wet or dry etching. This applies the semiconductor microfabrication technology to create microchannels having the shapes and sizes suitable for the form of red blood cell, white blood cell and blood platelet on the substrate with high accuracy. This technique enables to design the minute width and length ratio, interval and so on in accordance with the purpose of usage and also allows direct observation of actual flow in a flow channel through a transparent plate.

A technique described in Japanese Patent No. 2532707 uses a technique of leading blood sample from a large flow channel into a micro channel by a hydrostatic pressure difference or concentration difference of biologically active substance, thereby enabling measurement on a sufficient number of blood cells even with a small amount of blood sample by a significantly large number of blood cells contained therein.

However, the semiconductor microfabrication technology that micro-fabricates flow channels on a silicon substrate by wet or dry etching has practical drawbacks including: (1) high material cost of a silicon substrate, (2) high processing cost due to photolithography performed on each substrate, (3) varying dimensional accuracy of microchannels of each substrate and (4) no incinerability.

### SUMMARY OF THE INVENTION

An object of the present invention is to provide a resin microchannel array suitable for health care and diagnosis and treatment of disease, a manufacturing method of the resin microchannel array, and a blood test method.

According to the present invention, for achieving the above-mentioned object, there is provided a resin microchannel array which includes a first substrate having a plurality of depressions, each depression having an inlet port at one end and an outlet port at another end, and walls sectioning the depressions, each wall having a micro groove connecting the depressions, and a second substrate having a flat surface bonded or pressure-contacted to a surface of the first substrate, wherein spaces created by the depressions and the grooves in a bonded or pressure contacted part between the first substrate and the second substrate serve as flow channels.

In the above resin microchannel array, a passing resistance of the flow channel for each blood cell may be varied by setting either or all of the width, depth or shape of the groove to be the same as either of a red blood cell, a white blood cell and a blood platelet, or to be larger or smaller than those, or by placing a plurality of kinds, or a blood cell capable of passing through the flow channel created by the groove may be limited.

In the above resin microchannel array, each of a width and a depth of the groove is preferably within a range of 1 to 50 μm, and a ratio of the width and the depth of the flow channel is preferably within a range of 1:10 to 10:1.

In the above resin microchannel array, a contact angle of the resin microchannel array with respect to water is preferably from 0.5° to 70°. This structure is suitable for a blood sample to flow through a microchannel.

The present invention can thereby provide a resin microchannel array suitable for health care and diagnosis and treatment of disease, a manufacturing method of the resin microchannel array, and a blood test method.

The above and other objects, features and advantages of the present invention will become more fully understood from the detailed description given hereinbelow and the accompanying drawings which are given by way of illustration only, and thus are not to be considered as limiting the present invention.

### BRIEF DESCRIPTION OF THE DRAWINGS

FIGS. 1A to 1H are pattern diagrams showing the process steps of manufacturing a resin microchannel array according to the present invention.

FIGS. 2A and 2B are outline views of a resin microchannel array manufactured by the process shown in FIGS. 1A to 1H;

FIGS. 3A and 3B are detailed views of the outline of the resin microchannel array manufactured by the process shown in FIGS. 1A to 1H;

FIG. 4 is a view showing the structure of a microgroove connecting a wall and a depression of the resin microchannel array manufactured by the process shown in FIGS. 1A to 1H;

FIG. 5 is a view showing the structure of a microgroove connecting a wall and a depression of the resin microchannel array manufactured by the process shown in FIGS. 1A to 1H;

FIG. 6 is a view showing the structure of a microgroove connecting a wall and a depression of the resin microchannel array manufactured by the process shown in FIGS. 1A to 1H;

FIG. 7 is a view showing the structure of a microgroove connecting a wall and a depression of the resin microchannel array manufactured by the process shown in FIGS. 1A to 1H;

FIG. 8A and 8B are detailed views of the outline of a resin microchannel array manufactured by the process shown in FIGS. 1A to 1H;

FIG. 9 is a view showing the structure of a microgroove of a resin microchannel array manufactured by the process shown in FIGS. 1A to 1H;

FIG. 10 is a detailed view of the outline of a resin microchannel array manufactured by the process shown in FIGS. 1A to 1H;

FIG. 11 is an electron microgram of a microphase-separated structure by TEM; and

FIG. 12 shows an image observed optically with a CCD camera in an example.

### DESCRIPTION OF THE PREFERRED EMBODIMENTS

The present invention is described hereinafter in detail.

Blood is classified broadly into blood cell (formed) elements and blood plasma (fluid) elements. The percentage of the blood cell elements is about 40% to 45% and that of the blood plasma elements is about 55% to 60%. The blood cell elements are composed of about 96% of red blood cells and about 4% of white blood cells and blood platelets. A red blood cell has a diameter of about 7 to 8 μm, a white blood cell has a diameter of about 12 to 14 μm, and a blood platelet has a diameter of about 3 μm.

A blood test method according to the present invention uses a resin microchannel array having a first substrate and a second substrate bonded or pressure contacted to each other. The first substrate has a plurality of depressions each of which has an inlet port at one end and an outlet port at the other, and walls which section the depressions and each of which has a microgroove to connect the depressions. The second substrate has a flat surface that is bonded or pressure contacted to the surface of the first substrate. The resin microchannel array uses spaces that are created by the depressions and the microgrooves in the bonded or pressure contacted part between the first substrate and the second substrate as flow channels.

A blood test that is performed by using the flow channels created by the grooves is described below. The white blood cell activity refers to a synthesis of various reactions such as migration, phagocytosis and biologically active substance secretion, and the contraction and movement of contractile proteins in cells are concerned with each reaction. On the other hand, the active or passive ability of a white blood cell to pass through a flow channel including the flow channel obstruction significantly varies by the contraction or movement state of contractile proteins in the cell. Therefore, the active or passive flow channel passing ability of a white blood cell or the flow channel obstruction serve as appropriate indications of the white blood cell activity.

The aggregation of blood platelet is also the reaction that is based on the contraction and movement of contractile proteins in a cell. Therefore, the flow channel passing ability of a blood platelet or the flow channel obstruction due to blood platelet aggregate also serve as appropriate indications. It is also feasible for the white blood cells and blood platelets to use a change in flow channel passing ability including flow channel obstruction after providing stimulus with a certain amount of biologically active substance as an indication.

This method that lets blood sample through a flow channel of a large depression and a micro flow channel allows a large part of the sample to flow through the large channel and a small part of the sample to flow through the microchannel.

Therefore, in a micro flow channel whose inlet port has a shape in accordance with a red blood cell, even if a formed element that is larger than a white blood cell or a red blood cell, which is a blood platelet aggregate for example, comes close to the inlet port, it cannot flows into the channel but is drifted away from the inlet port by the mainstream of the blood sample.

This prevents a formed element larger than a white blood cell or a red blood cell from obstructing the flow channel. Though it cannot avoid inflow of a blood platelet smaller than a red blood cell, the blood platelet does not obstruct the passing of a red blood cell. Similarly, in a flow channel whose inlet port has a shape in accordance with a white blood cell, though a red blood cell and a blood platelet freely pass through the channel, they do not affect the passing of a white blood cell.

It is possible to selectively measure the flow channel passing ability including the channel obstruction due to blood cells under test while preventing the inflow of a blood cell or a formed element having a larger diameter by setting appropriate way of flowing a blood sample, shape of a flow channel inlet port, width and depth of a flow channel, and way of measuring. Further, it is possible to measure the red blood cells, white blood cells and blood platelets in a blood sample simultaneously and rapidly by arranging in parallel three kinds of flow channels respectively suitable for red blood cells, white blood cells and blood platelets and letting a blood cell flow through each of them.

The width and depth of a flow channel are preferably selected from the range of 1 to 50 μm and more preferably from 1 to 20 μm according to a blood cell element to be tested. The ratio of the width and depth of the flow channel is preferably selected from the range of 1:10 to 10:1 according to the shape and deformability of a blood cell element to be tested.

In order for the space created by the depression and groove of a resin microchannel array to serve as a flow channel, it is preferred that a difference in wettability is small between the resin microchannel array and a water-type fluid to be used, such as saline, blood cell and reagent. A large wettability difference can cause that the water-type fluid does not flow through the flow channel. Further, it causes entry of air bubbles when filling the flow channel with saline, for example, before blood test, thus failing to maintain the same discrete value of a passing time of a blood cell element to be tested.

Further, since cells are normally subject to immobilization onto a hydrophobic surface, it is likely in blood cells that blood cell elements are attached to a flow channel and cease to flow, which significantly affects the blood test.

It is thus required that the contact angle of the resin microchannel array surface with respect to water is small. Generally-used thermoplastic resin such as polymethyl methacrylate normally has a relatively large contact angle with respect to water (for example, about 68° with polymethyl methacrylate resin, about 70° with polycarbonate resin, and 84° with polystyrene resin). It is therefore necessary to reduce the contact angle to the range from 0.5° to 70°.

Techniques to modify the wettability of a plastic surface are classified broadly into chemical treatment techniques and physical treatment techniques. The chemical treatment techniques include chemical treatment, solvent treatment, coupling agent treatment, monomer coating, polymer coating, steam treatment, surface grafting, electrochemical treatment, anodic oxidation and so on. The physical treatment techniques include ultraviolet irradiation treatment, plasma contact treatment, plasma jet treatment, plasma polymerization treatment, ion beam treatment, mechanical treatment and so on.

Some of the above modification techniques develop adhesive properties in addition to hydrophilic properties of the thermoplastic resin surface. Since this may be unfavorable for maintaining a large number of micro flow channel patterns of a resin microchannel array, it is necessary to select a modification technique in accordance with a required contact angle. Examples of applicable modification techniques are described below.

The chemical treatment techniques include inorganic and organic material coating. This technique coats hydrophilic polymer such as polyvinyl alcohol by dipping, spin coating and so on and sufficiently dry it for use. If the hydrophobic property of a resin microchannel array is high, a uniform coating film thickness may not be obtained to cause variation in modification effects, and it is thus required to select an appropriate coating material. An example of the material that can be coated onto a hydrophobic surface is Lipidure-PMB, which is copolymer of MPC polymer having phospholipid polar group and butyl acrylate, available from NOF CORPORATION.

It provides modification effects with a relatively simple process without the need for a large size system, thus allowing cost reduction. However, since ultrasonic cleaning or the like can deteriorate the modification effects, it is preferred to repeat coating or use it for disposable applications.

The chemical treatment techniques also include steaming and, particularly, vapor deposition. The vapor deposition is one of inorganic thin film deposition techniques, which heats and vaporizes a substance to be formed into a thin film in vacuum (with a pressure of 10-2 Pa or lower) and deposits the vapor on an appropriate substrate surface. It enables processing at a relatively low degree of vacuum without the need for a large size system, thus allowing cost reduction.

The chemical treatment techniques also include plasma treatment, and particularly sputtering. The sputtering accelerates the positive ion generated by low-pressure grow discharge in electric field and make it collide against a cathode so that the substance on the cathode comes out and is deposited on the anode. The sputtering can deposit various kinds of materials, and deposition of an inorganic material such as SiO₂ and Si₃N₄ for 10 nm to 300 nm allows hydrophilization of a material surface.

This is also effective for a plurality of uses by repetitive ultrasonic cleaning and so on in that it enables sustainable effects and provides repeatable test results. Further, it has no effluent and is compatible with cytotoxicity that is required for bioengineering applications and so on. The sputtering enables to uniformize the thickness of a deposition film and, deposition of SiO₂ film with 10 to 50 nm in thickness allows achieving both transparency and hydrophilization.

When depositing an inorganic film on a resin microchannel array, sufficient degassing is required before sputtering in order to avoid that the resin microchannel array discharges absorbed moisture during sputtering to cause reduction of adhesion with the inorganic film. Other techniques for improving the adhesion of the resin surface and the inorganic film involve performing etching with argon gas or the like on the surface of a resin microchannel array, and depositing inorganic material with high adhesion, such as chromium, and then depositing a desired inorganic film. The sputtering requires about 50° C. to 110° C. heat-resistant temperature, and therefore it is essential to select the conditions such as (1) selecting polycarbonate or the like having a glass transfer temperature of higher than above temperature and (2) reducing a sputtering processing time (reducing a film thickness).

On the other hand, the physical treatment techniques include plasma treatment, particularly implantation. In the implantation, molecules are activated by plasma and radicals generated on a polymer surface recombine to form a new functional group on the polymer surface. The introduction of the functional group creates the polymer surface having a new property.

Another plasma treatment is plasma polymerization treatment. This technique vaporizes organic material that serves as raw material of polymeric material for vapor phase transition and then activates the organic material by electron collision excitation in plasma to cause polymerization reaction, thereby forming polymer coating on the substrate. The plasma polymerization method eliminates the need for solvent that can be impurity since it uses vaporized material molecule and allows easy control of a film thickness. Further, since there is no remaining monomer, it is compatible with cytotoxicity that is required for bioengineering applications and so on. The plasma polymerization treatment raises polymerization reaction by activating the organic material with electron collision excitation in plasma; on the other hand, it is vapor deposition polymerization that raises polymerization reaction by heat.

The physical treatment techniques also include UV treatment, particularly excimer UV treatment. In the hydrophilization of a thermoplastic resin, it requires a low heat-resistance temperature and it is thus applicable to polymethyl methacrylate with a glass transfer temperature of 100° C.

The excimer UV treatment uses an excimer lamp using discharge gas such as argon, krypton, and xenon for irradiation of ultraviolet light with the center emission wavelength of 120 nm to 310 nm. By the irradiation of the high energy ultraviolet light, the molecules on the resin surface dissociates and a light hydrogen atom is easily drawn to create a highly hydrophilic functional group such as OH, thereby increasing the wettability of the surface. This technique increases not only hydrophilic properties but also adhesive properties as the amount of UV exposure increases, which is sometimes unfavorable for maintaining a large number of micro groove patterns. It is therefore necessary to select the amount of exposure in accordance with a required contact angle.

Another method for hydrophilization is to use a vinyl acetate resin (product name: “Exceval”) available from KURARAY, CO., LTD, polyvinyl butyral resin and so on as a molded material. In order to maintain a micro flow channel form, it is necessary to use water-type fluid at a temperature of 70° C. or lower and avoid long time immersion in water.

The contact angle of the surface of a resin microchannel array with respect to water is preferably from 0.5°0 to 70° and more preferably 1° to 50° in order to smoothly lead a blood sample into a microchannel and obtain anti-adhesion property of a blood cell. If the contact angle is not within this range, it is difficult to lead a blood sample into a microchannel and the aggregate that is created due to attachment of the blood cells hinders obtaining stable data in the measurement of passing time of blood cells or the like. It is therefore preferred that the contact angle is within the above range.

The above technology may be applied not only to the resin microchannel array but also to a silicon plate that is manufactured by using semiconductor processing technology.

The surface property modification and hydrophilization techniques described above are useful in the biotechnology field as well. The research on cell growth and cell organization using various kinds of cells forms a minute pit and projection pattern on a plate and observes and evaluates the process of the growth and differentiation of cells in the minute spatial structure. It is preferred that the contact angle of the plate surface with respect to water is from 0.5° to 70° just like the resin microchannel array in order to eliminate the entry of air babbles and let water-type culture solution flow through the minute pit and projection pattern on the plate.

The blood platelet adhesiveness of the resin microchannel array surface is described below. Since blood cells are normally subject to immobilization onto a hydrophobic surface, it is sometimes necessary to hydrophilize the surface and also suppress the adhesion of blood platelets having blood coagulability to suppress generation of an aggregate. When blood and material contact to each other, blood platelet and protein are absorbed. On the surface of the blood platelet, activation such as discharge of inner substance by deformation or the like occurs to form an aggregate of blood elements. In order to increase the repeatability of data such as passing time measurement of blood cells, it is necessary in some cases to suppress the blood platelet adhesiveness.

A first material for preventing blood coagulation is a material containing heparin that is medical agent to prevent blood coagulation. A second is urokinase that is immobilized enzyme. A third is a material to prevent blood platelets and proteins in blood from attaching onto the surface. The surface of this material is coated with macromolecule with a high water content such as polyvinyl alcohol, acrylamide and polyethylene glycol. A fourth is a material to prevent the activation of blood platelets. This is the material having the surface structure of microphase-separated structure.

The separation size of the fourth microphase-separated structure has a uniform microdomain structure in the range from 20 nm to 20 μm. The suppression of the blood platelet adhesion by the microphase separation is possible by the combinations of amorphous and nonamorphous, hydrophilicity and hydrophobicity, crystal and noncrystal, glass and fluid, and so on. Materials include a copolymer of HEMA-styrene and HEMA-butadiene, a block copolymer of hydrophilic PHENA and hydrophobic styrene, a blend of crystalline Nylon-610 noncrystalline polypropylene oxide, and so on.

By forming a narrow pit and projection pattern in a microchannel, it is possible to achieve highly accurate blood testing. Forming the pit and projection pattern in the same channel enables not only the tracking of the blood cells passing therethrough but also the tracking of a change occurring in the passing process. The allocated way of each formed element of between different flow channels and the distributed state of each formed element in the same flow channel can serve as new indications.

For example, when measuring the activity of white blood cells by the speed of deformedly passing through the microchannel, the number of cells, deformability and so on, forming a plurality of projection patterns in the flow channel, not merely reducing the width and depth of flow channels, enables to clarify the difference between samples. The narrow pit and projection pattern is also effective for the case of immobilizing particular blood cells in the flow channel and performing optical detection. For example, when immobilizing a white cell blood with a diameter of about 12 μm, forming a narrow part with a width of 6 μm in a flow channel with a width of 12 μm and depth of 12 μm allows red blood cells and blood platelets to pass through while holding white blood cells.

It may be possible to form a minute pit and projection pattern having a width of as small as 1 μm by using a stepper, which is a reduction exposure machine, in the exposure process when producing a metal structure to serve as a master, for example. However, since a mask used for the exposure can be expensive in this case, and therefore it is preferred to select the size of the pit and projection pattern according to production costs and intended use.

Since the depth of a pit differs by a multistep pattern, it is possible to clarify the difference between samples in the measurement of blood cells about the speed of deformedly passing through the microchannel, the number of cells, deformability and so on. The blood sample flowing from an inlet port is led to the microchannel formed on the wall portion through the depression.

The depth and width required for leading a blood sample is preferably at least 30 μm and more preferably 80 μm. For example, if the depth and width of the depression is 80 μm and the depth and width of the flow channel is 5 μm, a blood sample is lead from a large space into an extremely narrow space, and a difference between samples may be difficult to find due to a change in the activity of blood platelets and so on even with a blood sample in mean state.

Therefore, the depth of depression is preferably in a multistep form such as 30 μm, 50 μm and 80 μm, for example, just like human blood capillary. The manufacturing method according to this invention produces a metal structure to serve as a master plate, and forms a plurality of resin microchannel arrays with high accuracy and high repeatability from one metal structure.

The manufacturing of a silicon plate by etching with use of the semiconductor processing technology needs to perform the number of etching processes according the number of steps to be needed, which causes variation in processing accuracy and high costs. The manufacturing method of the present invention, on the other hand, achieves both high accuracy and low costs by using a metal structure that satisfies the dimensional accuracy.

The resin microchannel array can be incinerated as infectious waste, just like thermoplastic resin such as a blood circuit used for blood purification treatment including artificial dialysis and plasma exchange. A silicon plate formed by a conventional etching process is inorganic material and thus incinerable. The landfill disposal as industrial waste requires sterilization and thus results in high costs. This is also against the increasing awareness of environmental issues.

On the other hand, the resin microchannel array of this invention can cope with increase in the number of wastes accompanying future increase in disposable products because of its incinerability. Further, forming a substrate to be overlapped with resin eliminates the need for separation so as to allow incineration all together. Furthermore, use of thermoplastic resin not containing halogen, such as polymethyl methacrylate, prevents generation of harmful dioxin and allows easy incineration with an incinerator at a temperature normally used for the incineration of domestic waste, and enables reuse as heat resource.

In the case of using optical detection system for blood test, when performing observation using a CCD camera or the like, it is necessary that either or both of the resin microchannel array and an overlap substrate is transparent for reflection light or transmission light measurement. For the reflection light measurement, the substrate on the optical system side is transparent and the substrate on the opposite side is opaque. To make an opaque substrate, it is feasible to select opaque grade when selecting material or to deposit inorganic film such as aluminum on the front or rear surface of a transparent substrate by deposition, for example.

It is thereby possible to directly observe the flow channel through the transparent substrate and take appropriate actions such as adjusting a flow speed and stopping a flow. Preferred optical property to provide transparency is that light transmittance is 80% or higher and haze is 10% or lower in a substrate with a thickness of 1 mm. Further, in the optical detection system, it is preferred to use material in accordance with light wavelength to be used, such as material not containing ultraviolet absorbent or material not having ring system in molecular structure.

A step of forming a resist pattern on a substrate, a step of forming a metal structure by depositing a metal according to the resist pattern formed on the substrate, and a step of forming a resin microchannel array by using the metal structure are described below.

In the method of manufacturing a resin microchannel array according to this embodiment, a resist pattern is formed by:
(i) formation of a first resist layer on a substrate;
(ii) positioning of the substrate and a mask A;
(iii) exposure of the first resist layer with the use of the first mask;
(iv) heat treatment on the first resist layer;
(v) formation of a second resist layer on the first resist layer;
(vi) positioning of the substrate and a mask B;
(vii) exposure of the second resist layer with the use of the mask B;
(viii) heat treatment on the second resist layer; and
(ix) development of the resist layers.

Further, according to the resist pattern thus formed, a metal structure is deposited on the substrate by plating. Then, a resin molded product is formed by using the metal structure as a mold, thereby producing a resin microchannel array.

The resist pattern formation step is described in further detail below. To form a micro groove with the depth of 10 μm and a depression with the depth of 80 μm on a substrate, for example, a first resist layer (80 μm in thickness) is deposited and a second resist layer (20 μm in thickness) is deposited thereon, and each layer is exposed or exposed and heat-treated.

In the development process, a pattern with the depth of 10 μm to serve as the second resist layer, is obtained firstly. Then, a pattern with the depth of 80 μm to serve as first resist layer is obtained. In order to prevent the 10 μm pattern of the second resist layer from being dissolved or distorted by a developer in the formation of the 80 μm pattern, it is required to control the solubility of each layer in the developer. When forming the resist layer by spin coating, it is possible to develop alkali resistance by adjusting a baking (solvent drying) time of the second resist layer.

One technique for developing the alkali resistance of photodegradable positive resist is to increase a baking time (solvent drying time) so as to harden the resist. The baking time of the resist is normally adjusted according to the thickness of layer, the density of solvent such as thinner, and the sensitivity. Increasing the baking time can develop the alkali resistance.

Overbaking of the first resist layer hardens the resist too much, making it difficult to dissolve an exposed part and form a pattern in the subsequent development step. Thus, it is preferred to adjust baking conditions by reducing the baking time and so on. Equipment used for the baking is not particularly limited as long as it can dry a solvent, including an oven, a hot plate, a hot-air dryer, and so on.

Since the development of the alkali resistance is limited compared to photocrosslinkable negative resist, the combined thickness of each resist layer is preferably 5 to 200 μm, and more preferably, 10 to 100 μm.

Besides the optimization of the baking time, another method for developing the alkali resistance of the photocrosslinkable negative resist is optimization of crosslink density. Normally, the crosslink density of the negative resist can be adjusted by the exposure amount. In the case of chemical amplification resist, it can be adjusted by the exposure amount and the heat-treatment time. The alkali resistance can be developed by increasing the exposure amount or the heat-treatment time. When using the photocrosslinkable negative resist, the combined thickness of each resist layer is preferably 5 to 500 μm, and more preferably, 10 to 300 μm.

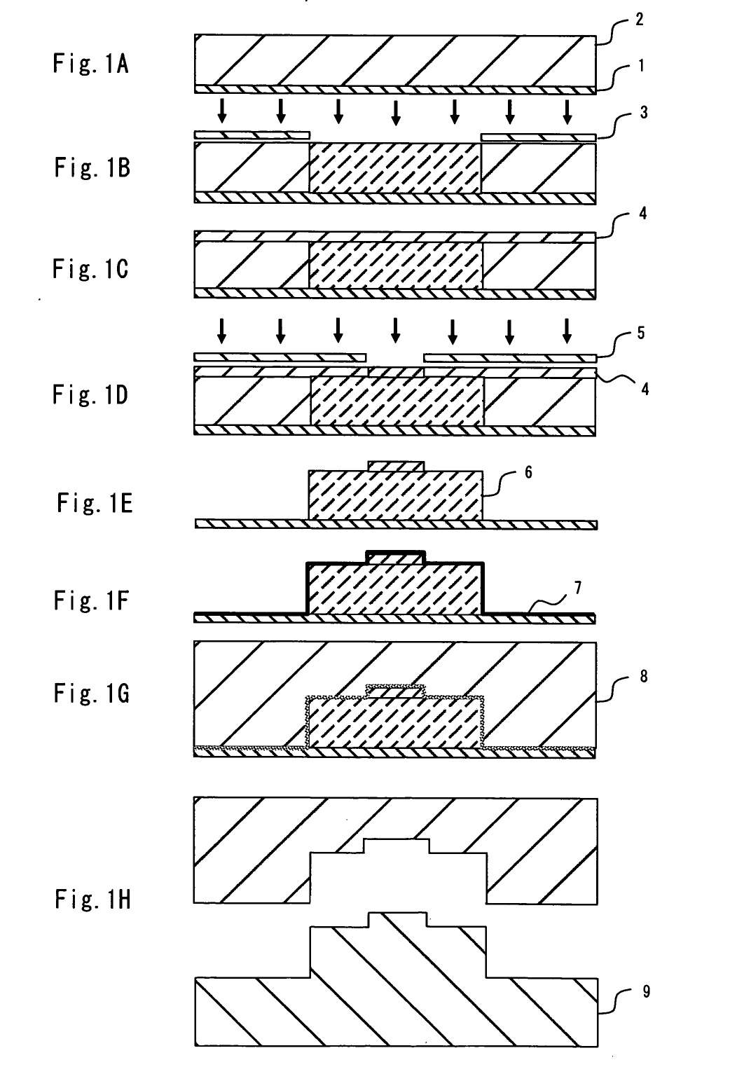

(i) The formation of the first resist layer 2 on the substrate 1 is described below. FIG. 1A shows the first resist layer 2 formed on the substrate 1. The flatness of a resin microchannel array obtained by the molded product formation step is determined by the step of forming the first resist layer 2 on the substrate 1. Thus, the flatness when the first resist layer 2 is deposited on the substrate 1 is reflected in the flatness of the metal structure and the resin microchannel array eventually.

Though a technique to form the first resist layer 2 on the substrate 1 is not limited in any way, spin coating, dip coating, roll coating, and dry film resist lamination are generally used. Particularly, the spin coating is a technique to deposit a resist on a spinning glass substrate and it has an advantage of very flat coating of the resist on a glass substrate with the diameter of more than 300 mm in diameter. The spin coating is thus preferred for use to achieve high flatness.

There are two types of resists that may be used for the first resist layer 2: positive resist and negative resists. Since the depth of focus on the resist changes depending on the resist sensitivity and exposure conditions, when using a UV exposure system, for example, it is preferred to select an exposure time and a UV output level according to the type, thickness, and sensitivity of the resist. When using the wet resist, techniques for obtaining a desired resist thickness with the use of the spin coating, for example includes changing the spin coating rotation speed and adjusting the viscosity.

The technique of changing the spin coating rotation speed obtains a given resist thickness by controlling the rotation speed of a spin coater. The technique of adjusting the viscosity controls the resist viscosity according to the flatness level since the degradation of flatness can occur if the resist is too thick or the resist deposition area is too large.

In the spin coating, for example, the thickness of the resist layer deposited at a time is preferably 10 to 50 μm, more preferably 20 to 50 μm, to maintain high flatness. Obtaining a desired resist layer thickness while retaining high flatness is achieved by forming a plurality of resist layers.

When photodegradable positive resist is used for the first resist layer 2, if a baking time (solvent drying) is too long, the resist hardens too much, making it difficult to form a pattern in the subsequent development step. Thus, it is preferred to select appropriate baking conditions by reducing the baking time and so on if the resist thickness is less than 100 μm.

(ii) The positioning of the substrate 1 and the mask A 3 is described below. In order to make the positional relationship between the pattern of the first resist layer and the pattern of the second resist layer as designed, it is necessary to perform accurate positioning in the exposure using the mask A 3. The positioning may be made by providing cutting in the corresponding positions of the substrate 1 and the mask A 3 and fixing them with pins, reading the positions by laser interferometry, creating position marks in the corresponding positions of the substrate 1 and the mask A 3 and performing positioning with an optical microscope, and so on.

The method of performing positioning with an optical microscope may create a position mark on the substrate by photolithography and create a position mark on the mask A 3 by laser beam equipment, for example. This method is effective in that the accuracy within 5 μm can be easily obtained by manual operation using the optical microscope.

(iii) The exposure of the first resist layer 2 with the use of the mask A 3 is described below. The mask A 3 used in the step shown in FIG. 1B is not limited in any way. For example, an emulsion mask, a chrome mask, and so on may be used. In the resist pattern formation step, the size and accuracy depends on the mask A to be used. The size and accuracy are reflected in the resin molded product. Hence, to obtain a resin microchannel array with a prescribed size and accuracy, it is necessary to specify the size and accuracy of the mask A. A technique to increase the accuracy of the mask A 3 is not limited in any way. For example, one technique is to replace the laser light used for the pattern formation of the mask A 3 with the light having a shorter wavelength. This technique, however, requires high facility costs, resulting in higher fabrication costs of the mask A 3. It is thus preferred to specify the mask accuracy according to the accuracy level required for practical use of the resin microchannel array.

The material of the mask A 3 is preferably quartz glass in terms of temperature expansion coefficient and UV light transmission and absorption characteristics; however, since it is relatively expensive, the material is preferably selected according to the accuracy level required for practical use of the resin molded product. In order to obtain a prescribed structure with different depths or heights or a structure in which the first resist pattern and the second resist pattern are different, it is necessary to ensure the design of the patterns (transmitting/shielding parts) of the masks that are used for the exposure of the first resist layer 2 and the second resist layer 4. One approach to achieve this is to perform simulation by using CAE analysis software.

The light used for the exposure is preferably ultraviolet light or laser light for low facility costs. Though synchrotron radiation makes deep exposure, it requires high facility costs and thus substantially increases the price of a resin microchannel array, and therefore it is not industrially practical.

Since exposure conditions such as exposure time and intensity vary by material, thickness, and so on of the first resist layer 2, they are preferably adjusted according to the pattern to be formed. The adjustment of the exposure conditions is important since it affects the accuracy and the sizes of a pattern such as the width and height of a flow channel, and the interval, width (or diameter), and height of a reservoir. Further, since the depth of focus changes depending on the resist type, when using the UV exposure system, for example, it is preferred to select an exposure time and a UV output level according to the thickness and sensitivity of the resist.

(iv) The heat-treatment of the first resist layer 2 is described below. Annealing is known as a heat-treatment after the exposure to correct the shape of the resist pattern. In this case, it aims at chemical crosslinking and is used only when a chemical amplification negative resist is used. The chemical amplification negative resist is mainly composed of two- or three-component system. For example, a terminal epoxy group at an end of a chemical structure is ring-opened by exposure light and crosslinking reaction occurs by the heat-treatment. If the layer thickness is 100 μm, for example, the crosslinking reaction progresses in several minutes by the heat-treatment with the temperature of 100° C.

Excessive heat-treatment on the first resist layer 2 makes it difficult to dissolve a non-crosslinked part to form a pattern in the subsequent development step. Thus, if the resist thickness is less than 100 μm, it is preferred to adjust the processing by reducing a heat-treatment time, performing the heat-treatment only on the second resist layer 4 formed later and so on.

(v) The formation of the second resist layer 4 on the first resist layer 2 is described below. FIG. 1C shows the state where the second resist layer 4 is formed. The second resist layer 4 may be formed by the same process as the formation of the first resist layer 2 explained in the step (a).

When forming a positive resist layer by the spin coating, increasing the baking time about 1.5 to 2 times longer than usual enables the development of alkali resistance. It is thereby possible to prevent the dissolution or distortion of the resist pattern of the second resist layer 4 at the completion of the development of the first resist layer 2 and the second resist layer 4.

(vi) The positioning of the substrate 1 and the mask B 5 is described below. The positioning of the substrate 1 and the mask B 5 is performed in the same manner as the positioning of the substrate 1 and the mask A 3 described in the step (ii).

(vii) The exposure of the second resist layer 4 with the mask B 5 is described below. The exposure of the second resist layer 4 with the mask B 5 is performed in the same manner as the exposure of the first resist layer 2 with the mask A 3 described in the step (iii). FIG. 1D shows the exposure of the second resist layer 4.

(vii) The heat-treatment of the second resist layer 4 is described below. The heat-treatment of the second resist layer 4 is basically the same as the heat-treatment of the first resist layer 2 described in the above step (iv). The heat-treatment of the second resist layer 4 is performed in order to avoid the dissolution or distortion of the pattern of the second resist layer 4 when the pattern of the first resist layer 2 is formed in the subsequent development step. The heat-treatment enhances the chemical crosslinking to increase the crosslink density, thereby developing the alkali resistance. The heat-treatment time for developing the alkali resistance is preferably selected according to the resist thickness from the range of 1.1 to 2.0 times longer than usual.

(ix) The development of the resist layers 2 and 4 is described below. The development in the step shown in FIG. 1E preferably uses a prescribed developer suitable for the resist to be used. It is preferred to adjust development conditions such as development time, development temperature, and developer density according to the resist thickness and pattern shape. For example, setting appropriate conditions is preferred since overlong development time causes the pattern to be larger than a predetermined size.

As the entire thickness of the resist layers 2 and 4 increases, the width (or diameter) of the top surface of the resist may become undesirably larger than that of the bottom of the resist in the development step. Thus, when forming a plurality of resist layers, it is preferred in some cases to form different resist with different sensitivity in each resist layer formation step. In this case, the sensitivity of the resist layer close to the top is set higher than that of the resist layer close to the bottom. Specifically, BMR C-1000PM manufactured by TOKYO OHKA KOGYO CO., LTD. may be used as the higher sensitivity resist and PMER-N-CA3000PM manufactured by TOKYO OHKA KOGYO CO., LTD. may be used as the lower sensitivity resist. It is also possible to adjust the sensitivity by changing the drying time of the resist. For example, in the case of using BMR C-1000PM manufactured by TOKYO OHKA KOGYO CO., LTD., drying of the first resist layer for 20 minutes at 110° C. and the second resist layer for 40 minutes at 110° C. in a resist drying operation after the spin coating allows the second resist layer to have the higher sensitivity.

Methods to increase the flatness accuracy of the top surface of the molded product or the bottom of the micro pattern include a method of changing the type of resist (negative or positive) used in the resist coating to apply the flatness of the glass surface and a method of polishing the surface of a metal structure.

In the case of forming a plurality of resist layers to obtain a desired pattern depth, it is feasible to perform the exposure and development of the plurality of resist layers at the same time. It is also feasible to form and expose one resist layer and further form and expose another resist layer, and then perform the development of the two resist layers at the same time.

The metal structure formation step is described herein in further detail. The metal structure formation step deposits a metal over the resist pattern formed by the resist pattern formation step to form an uneven surface of a metal structure in accordance with the resist pattern, thereby producing the metal structure.

This step first deposits a conductive layer 7 in accordance with the resist pattern as shown in FIG. 1F. Though a technique of forming the conductive layer 7 is not particularly limited, it is preferred to use vapor deposition, sputtering, and so on. A conductive material used for the conductive layer 7 may be gold, silver, platinum, copper, and aluminum, for example.

After forming the conductive layer 7, a metal is deposited in accordance with the pattern by plating, thereby forming the metal structure 8 as shown in FIG. 1G. A plating method for depositing the metal is not particularly limited, and electroplating or electroless plating may be used, for example. A metal used is not particularly restricted, and nickel, nickel and cobalt alloy, copper, or gold may be used, for example. Nickel is preferred since it is durable and less costly.

The metal structure 8 may be polished depending on its surface condition. In this case, to prevent contaminations from attaching to a product, it is preferred to perform ultrasonic cleaning after the polishing. Further, it is also feasible to perform surface treatment of the metal structure 8 with mold release agent or the like so as to improve the surface condition. The angle of gradient along the depth direction of the metal structure 8 is preferably 50° to 90°, and more preferably, 60° to 87°. The metal structure 8 deposited by plating is then separated from the resist pattern.

The molded product formation step is described hereinafter in further detail. The molded product formation step uses the metal structure 8 as a mold to form a resin molded product 9 as shown in FIG. 1H. Though a technique to form the resin molded product 9 is not particularly limited, injection molding, press molding, monomer casting, solution casting, or roll transfer by extrusion molding may be used, for example. The injection molding is preferred for its high productivity and pattern reproducibility.

The semiconductor microfabrication technique that uses silicon material has problems such as high material cost of a silicon substrate, high processing cost due to photolithography performed for each and every substrate, and varying dimensional accuracy of microchannels of each substrate. On the other hand, the formation of the resin molded product 9 by the injection molding with the use of the metal structure 8 having a prescribed size as a mold enables to reproduce the shape of the metal structure 8 into the resin molded product 9 with a high reproduction rate. This has advantages such as being suitable for cost reduction (commercial production) by the use of multipurpose resin material to reduce material costs, being capable of satisfying high dimensional accuracy, and so on.

The reproduction rate may be checked by using an optical microscope, a scanning electron microscope (SEM), a transmission electron microscope (TEM), a CCD camera and so on. By applying the quality control technique of optical discs, which has been actually put into commercial production, to the resin microchannel array, it is possible to manage and control various dimensional data, substrate flatness data, internal remaining stress data and so on based on standard deviation in lots of several tens of thousands.

In the case of producing the resin molded product 9 by using the metal structure 8 as a mold by the injection molding, for example, 10,000 to 50,000 pieces or even 200,000 pieces of resin molded products may be obtained with one metal structure 8. It is thus possible to largely eliminate the costs for producing the metal structures 8. Besides, one cycle of the injection molding takes only 5 to 30 seconds, being highly productive. The productivity further increases with the use of a mold capable of simultaneous production of a plurality of resin molded products in one injection molding cycle. In this molding process, the metal structure 8 may be used as. a metal mold; alternatively, the metal structure 8 may be placed inside a prepared metal mold.

A resin material used for the formation of the resin molded product 9 is not particularly limited. Acrylic resin, polylactide resin, polyglycolic acid resin, styrene resin, acrylic-styrene copolymer (MS resin), polycarbonate resin, polyester resin such as polyethylene terephthalate, polyvinyl alcohol resin, ethylene-vinyl alcohol copolymer, thermoplastic elastomer such as styrene elastomer, vinyl chloride resin, or silicone resin such as polydimethylsiloxane, vinyl acetate resin (product name: “Exceval”), polyvinyl butyral resin and so on may be used, for example.

The above resin may contain one or more than one agent of lubricant, light stabilizer, heat stabilizer, antifogging agent, pigment, flame retardant, antistatic agent, mold release agent, antiblocking agent, ultraviolet absorbent, antioxidant, and so on.

The minimum value of the flatness of the resin molded product 9 is preferably 1 μm to enable easy industrial reproduction. The maximum value of the flatness of the resin molded product 9 is preferably 200 μm in order not to cause a problem in the adhesion or overlapping of the molded product 9 to another substrate. The dimensional accuracy of the pattern of the resin molded product 9 is preferably within the range of ±0.5 to 10% to enable easy industrial reproduction.

The dimensional accuracy of the thickness of the resin molded product 9 is preferably within the range of ±0.5 to 10% to enable easy industrial reproduction. The thickness of the resin molded product 9 is not particularly specified, but it is preferably within the range of 0.2 to 10 mm to prevent breakage at removal in the injection molding, or breakage, deformation, or distortion during operation. The size of the resin molded product 9 is also not particularly specified, and it is preferably selected according to usage. For example, when forming the resist pattern by the lithography technique, if the resist layer is formed by spin coating, the diameter is preferably within 400 mm in diameter.

In the blood test, by letting saline, blood sample or reagent flow separately or simultaneously into a single or a plurality of the inlet ports of a resin microchannel array, it is possible to obtain test data according to each sample. Further, by placing a flow control system in either or both of the vicinity of the inlet port and the vicinity of the outlet port of a test device, an operator who conducts blood test can easily repeat an optimal flowing condition, thus allowing the improvement in blood test efficiency. The blood or its elements flowing through the resin microchannel array is recovered at the outlet port and then returned or sent to a different test system according to need.

An optical system that applies light to the inlet port and the outlet port of the depression connected through the flow channel or to the flow channel portion and the adjustment of a light amount reflected or transmitted by the flow channel enable to obtain more quantitative data. The optical systems may be a fluorescence microscope, a laser microscope, a laser scanner and so on. Activating the fluorescence of either each blood cell or fluid element with a fluorescent substance, or distinguishing the fluorescence intensity of each blood cell significantly facilitate differentiate between different kinds of blood cells and between a blood cell and a surrounding fluid. It is preferred to use system program with a computer in order to increase test points and accumulate and evaluate test data.

The blood test may measure a change in the number of each formed elements of blood at the inlet port and the outlet port of the depression connected through the flow channel or the obstruction state of the groove channel by each formed elements of blood, thereby obtaining the flowing characteristics or the activity of each formed elements of blood. Further, according to this test method, a person being tested with a high total cholesterol, for example, can visually observe the way that actual blood obstructs a microchannel, which is just like a capillary on the resin microchannel array. This provides a good opportunity for the person to recognize the need to improve eating habits, which are one of the factors to cause life-style related diseases. Therapeutics thereby actually contributes to increase the awareness of preventive medicine by way of such a visual observation.

The way that a blood sample flows in the blood test according to this embodiment also allows the measurement of migration of particular blood cells only by a difference in concentration of biologically active substances. Specifically, if a difference in concentration of biologically active substances is made instead of a hydrostatic pressure difference between the inlet port and the outlet port of the flow channel, only the blood cells capable of recognizing a concentration difference of the biologically active substances migrates into the flow channel. Measuring the number of cells and a passing time enables the blood test.

A difference between test substances may be checked also by conducting the blood test on a blood sample after exposure to a biologically active substance.

The blood test may use surface plasmon resonance (SPR). A detection system using SPR applies light onto a plate with a thin film such as a gold formed by deposition or the like and detects a change in permittivity on the surface of the thin film as a change in intensity of reflected light with high accuracy. The SPR device has been recently applied to the measurement of reaction and coupling between biomolecules and the kinetic analysis, which require extremely high sensitivity.

The blood test with the use of the resin microchannel array may also use the surface plasmon resonance for detection. Specifically, the test may form a thin film such as a gold on a resin microchannel array or an overlap substrate by deposition or the like, detect the activity of a white blood cell immobilized in the micro flow channel, for example, as a change in permittivity on the thin film surface (a change in reflected light intensity), and then convert the result into an electrical signal and amplifies the signal. It is thereby possible to measure a difference in the activity of each sample in numerical terms with high accuracy. The SPR sensor is capable of the measurement by specifying a part of a depression and a micro flow channel as a result of miniaturization by the semiconductor processing technology.

Further, the blood test may use a sensor for detecting an electric displacement electrochemically, such as a FET sensor, for example. The ion sensitive FET sensor coats the surface of a Si chip with SiO₂-Si₃N₄ film and amplifies a potential change that occurs by chemical species absorbed on the surface by using a field effect transistor (FET). The applied research to reaction between biomolecules, which requires extremely high sensitivity, has been developed, and a micro glucose sensor or the like has been introduced.

The blood test with the use of the resin microchannel array may also use the FET sensor for detection. For example, the test may fix the FET sensor and an electrode onto an overlap substrate, detect the activity of a white blood cell immobilized in the micro flow channel, for example, as a change in potential of the electrode surface, and electrically amplify the result. It is thereby possible to measure a difference in the activity of each sample in numerical terms with high accuracy.

Methods for positioning each substrate include a method of forming a pit and projection pattern on the front and rear surfaces of the substrates so that they are adhered with high positional accuracy when overlapped, a method of fixing the outer end portions of the substrates by jigs, a method of using positioning pins into through holes for fixation, a method of observing and adjusting the positions by using a CCD camera and a laser optical device and so on. The FET sensor is capable of the measurement by specifying a part of a depression and a micro flow channel as a result of miniaturization by the semiconductor processing technology. If the resin microchannel array is disposable and the overlap substrate is used repeatedly, it is possible to reduce costs for inspection.

The blood test may use a ultrasonic sensor for measurement. The applied research to reaction between biomolecules, which requires extremely high sensitivity, has been developed. The blood test with the use of the resin microchannel array may also use the ultrasonic sensor for detection. For example, the test may fix the ultrasonic sensor and an electrode onto an overlap substrate, detect the activity of a white blood cell immobilized in the micro flow channel, for example, as a slight frequency change, and convert the result into an electrical signal and amplifies the signal. It is thereby possible to measure a difference in the activity of each sample in numerical terms with high accuracy. The ultrasonic sensor is capable of the measurement by specifying a part of a depression and a micro flow channel as a result of miniaturization by the semiconductor processing technology. If the resin microchannel array is disposable and the overlap substrate is used repeatedly, it is possible to reduce costs for inspection.

### EXAMPLES

A method of producing a resin microchannel array according to the present invention is described hereinafter in further detail with reference to the drawings. Though the present invention is described in detail based on examples, it is not limited to these examples.

A method of producing a resin molded product according to the present invention is described with reference to the drawings. Referring first to FIG. 1A, the first resist coating on a substrate was performed by using an organic material (PMER N-CA3000PM manufactured by TOKYO OHKA KOGYO CO., LTD.).

Referring next to FIG. 1B, after the first resist layer formation, positioning of the substrate and a mask A having a desired mask pattern was performed.

After that, the first resist layer was exposed to UV light by a UV exposure system (PLA-501F manufactured by CANON INC. with the wavelength of 365 nm and the exposure dose of 300 mJ/cm²). The first resist layer was then heat-treated by using a hot plate at 100° C. for 4 minutes.

Referring then to FIG. 1C, the second resist coating on the substrate was performed by using an organic material (PMER N-CA3000PM manufactured by TOKYO OHKA KOGYO CO., LTD.).

Referring then to FIG. 1D, after the second resist layer formation, positioning of the substrate and a mask B with a desired mask pattern was performed.

After that, the second resist layer was exposed to UV light by a UV exposure system (PLA-501F manufactured by CANON INC. with the wavelength of 365 nm and the exposure dose of 100 mJ/cm²). The second resist layer was then heat-treated by using a hot plate at 100° C. for 8 minutes.

Referring then to FIG. 1E, development was performed on the substrate having the resist layers, thereby forming a resist pattern on the substrate (developer: PMER developer P-7G manufactured by TOKYO OHKA KOGYO CO., LTD.)

Referring to FIG. 1F, vapor deposition or sputtering was performed on the substrate with the resist pattern, thereby depositing a conductive layer formed of silver on the surface of the resist pattern. Instead of the silver, platinum, gold, copper, or the like may be deposited in this step.

Referring further to FIG. 1G, the substrate having the resist pattern was immersed in a plating solution for electroplating to form a metal structure (hereinafter referred to as the Nickel structure) in gaps in the resist pattern. Instead, copper, gold, or the like may be deposited in this step.

Referring finally to FIG. 1H, a plastic material was filled in the Ni structure by using the Nickel structure as a mold by injection molding. A plastic molded product was thereby produced. A material used for the injection molding was PARAPET GH-S, which is acryl manufactured by KURARAY, CO. LTD.

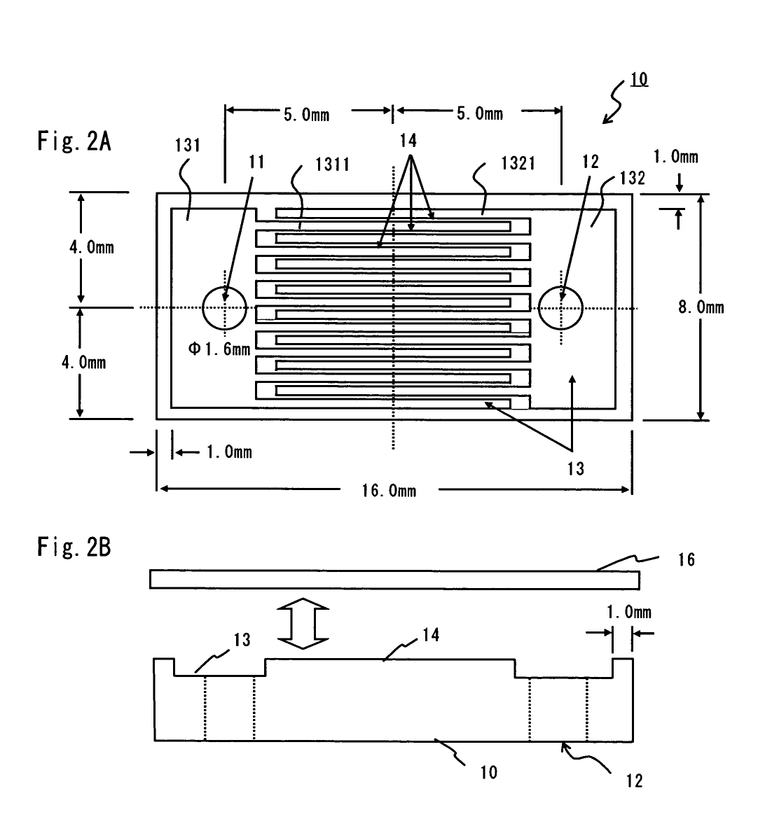

The shape of the resin microchannel array is described herein. The outer shape is a substrate with 16 mm in width, 8 mm in length, and 1.0 mm in thickness. The substrate has a through hole with a diameter of 1.6 mm as an inlet port at the left end and an outlet port at the right end. There are fifteen walls to section the depressions, and one groove has 340 micro grooves, thus having 5100 grooves in total. FIGS. 2A and 2B show the outline views. FIG. 2A is a top view of the resin microchannel array, and FIG. 2B is a sectional view of the resin microchannel array.

The resin microchannel array is composed of a first substrate 10 and a second substrate 16 overlapping with each other. The first substrate 10 has a depression 13. The depression 13 includes a rectangular depression 131 formed in the vicinity of one end and a rectangular depression 132 formed in the vicinity of the other end. A plurality of long depressions 1311 and 1321 are formed from the depressions 131 and 132 toward the center of the substrate 10. The long depressions are formed in such a way that the depression 1311 extended from the depression 131 and the depression 1321 extended from the depression 132 are arranged alternately. A wall 14 is created between the adjacent depressions. The wall 14 does not completely separate the adjacent depressions 1311 and 1321 but has a large number of minute grooves. In this example, one wall 14 has 340 minute grooves. The minute grooves connecting between the depressions 1311 and 1321 serve as flow channels.

The first substrate 10 has an inlet port 11 into which saline, blood sample or reagent flows. The inlet port 11 is a through hole in the depression 131 of the first substrate 10. The first substrate 10 also has an outlet port 12 at the position distant from the inlet port 11. The outlet port 12 is a through hole in the depression 132 of the first substrate 10. In this example, the inlet port 11 and the outlet port 12 are both cylindrical holes with a diameter of 1.6 mm.

As shown in FIG. 2B, the surface of the first substrate 10 having the depression 13 is overlapped with the second substrate 16, thereby creating a space between the depression 13 and the micro grooves, and the substrate 16.

When a blood sample or the like enters through the inlet port 11, it flows through the space of the depression 131 into the long depression 1311. Then, the blood sample or the like passes through the micro grooves formed between the depression 1311 and the depression 1321 to flow into the depression 1321. The white blood cells and blood platelets that are contained in the blood sample or the like passing through the micro grooves are observed. The blood sample or the like flowing from the depression 1321 into the depression 132 then flows out through the outlet port 12.

[Manufacture of Resin Substrate A]

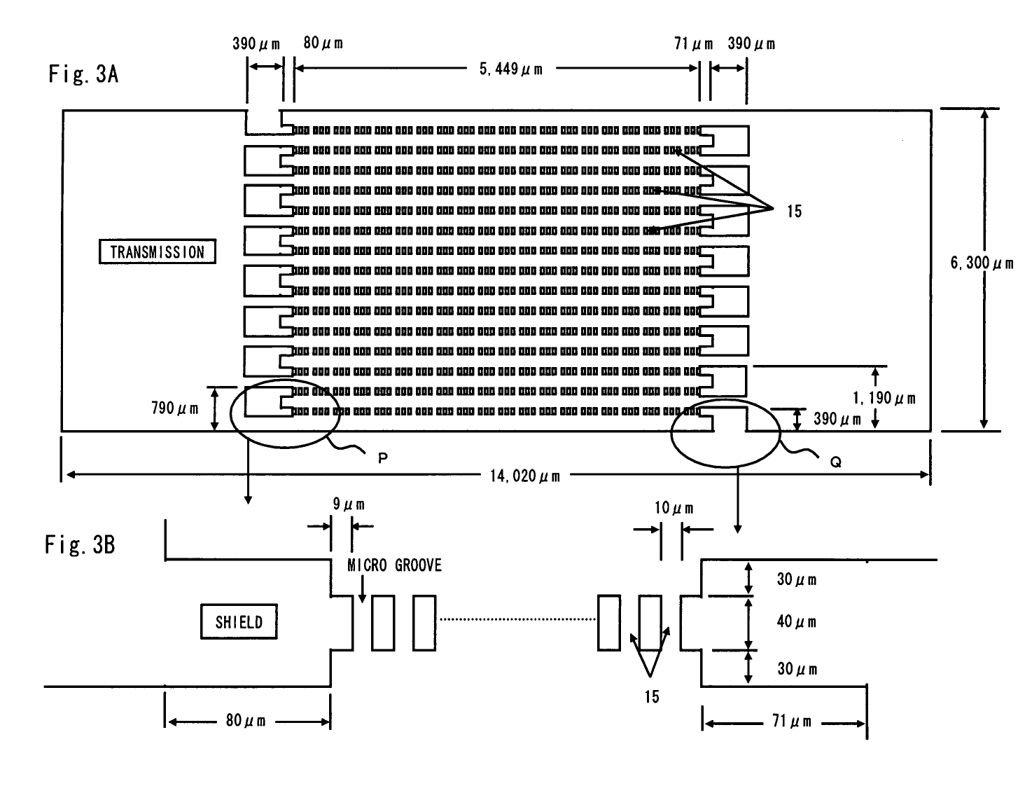

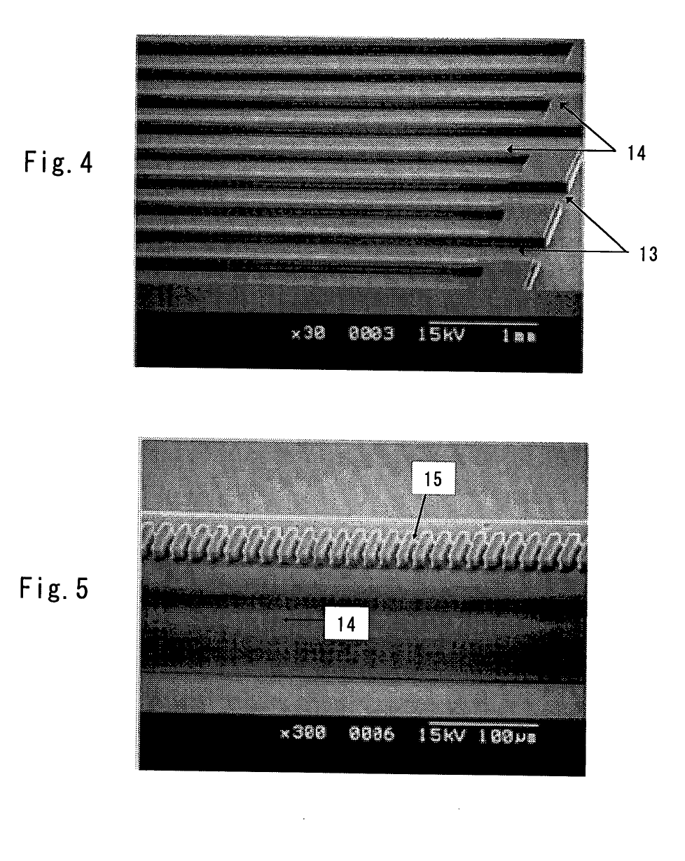

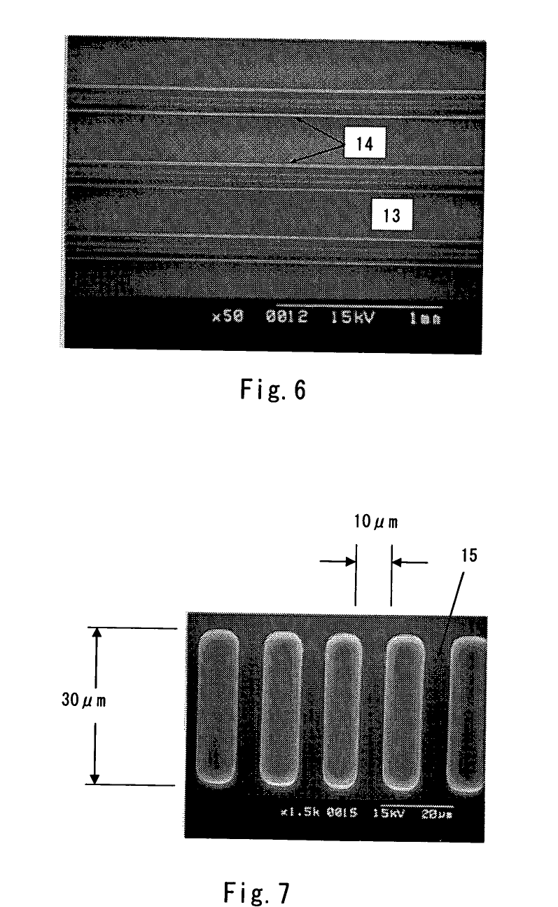

According to the molded product production process shown in FIGS. 1A to 1H, the resist coating was repeated twice to form the first resist layer and then exposure and heat-treatment were performed on each layer. Further, the resist coating was performed once again to form the second resist layer, and then the exposure and the heat-treatment were performed thereon. A resin microchannel array, as shown in FIG. 3A, having a substrate with 16 mm in width, 8 mm in length, and 1.0 mm in thickness on which a micro groove 15 with 10 μm in width and 7 μm in depth and a depression with 80 μm in depth were formed was thereby manufactured. FIG. 3B is an enlarged top view of the portion P and the portion Q in FIG. 3A. A contact angle with respect to water was measured in the air. The measurement with a contact angle measurement device (CA-DT/A manufactured by KYOWA INTERFACE SCIENCE CO., LTD.) resulted in 70°. FIGS. 4, 5, 6 and 7 show the structures of the walls that section the depressions and the micro grooves that connect between the depressions.

[Manufacture of Resin Substrate B]

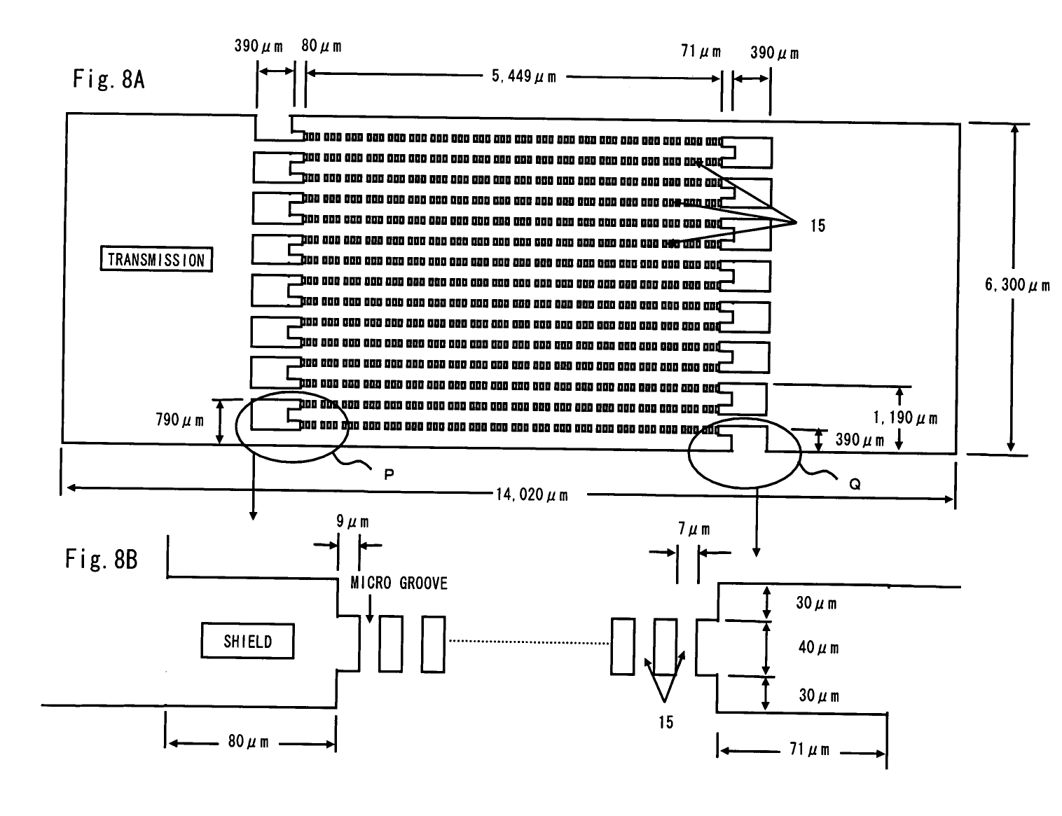

According to the molded product production process shown in FIGS. 1A to 1H, the resist coating was repeated twice to form the first resist layer and then exposure and heat-treatment were performed on each layer. Further, the resist coating was performed once again to form the second resist layer, and then the exposure and the heat-treatment were performed thereon. A resin microchannel array, as shown in FIG. 8A, having a substrate with 16 mm in width, 8 mm in length, and 1.0 mm in thickness on which a micro groove with 7 μm in width and 5 μm in depth and a depression with 80 μm in depth were formed was thereby manufactured. FIG. 8B is an enlarged top view of the portion P and the portion Q in FIG. 8A.

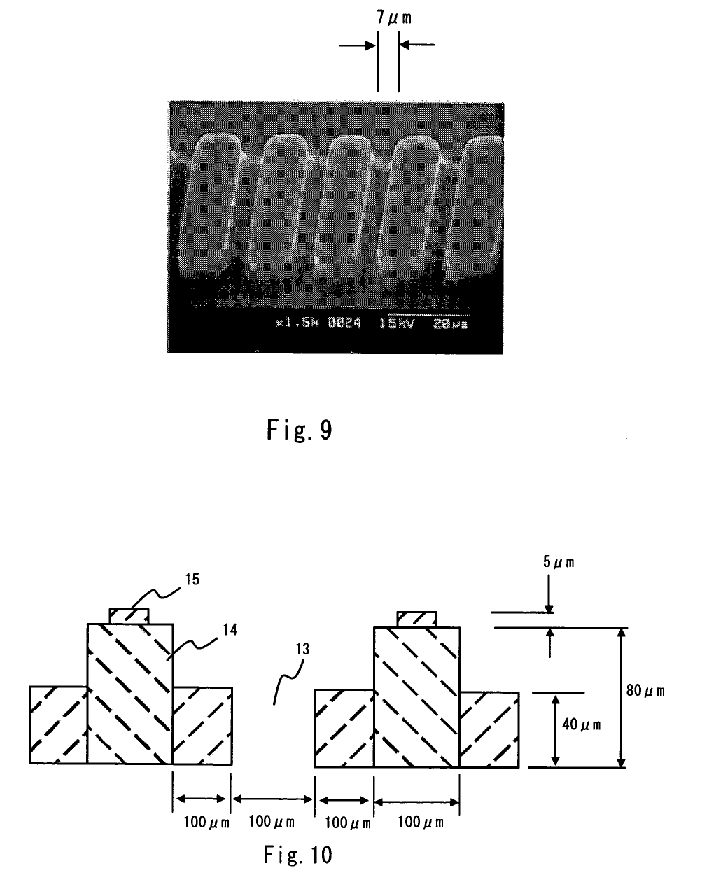

Surface modification by ultraviolet irradiation was performed on the manufactured microchannel array and the acrylic flat plate. Excimer light (172 nm) irradiation device (UER manufactured by USHIO INC.) was used for the irradiation of ultraviolet light for 60 seconds. Then, the contact angle with respect to water was measured as is the case with the resin substrate A, which resulted in 19°. FIG. 9 shows the structure of the groove formed in the wall.

[Manufacture of Resin Substrate C]

According to the molded product production process shown in FIGS. 1A to 1H, the resist coating was repeated twice to form the first resist layer and then exposure and heat-treatment were performed on each layer. Further, the resist coating was performed once again to form the second resist layer, and then the exposure and the heat-treatment were performed thereon. A resin microchannel array, as shown in FIG. 8A, having a substrate with 16 mm in width, 8 mm in length, and 1.0 mm in thickness on which a micro groove with 7 μm in width and 5 μm in depth and a depression with 80 μm in depth were formed was thereby manufactured.

Surface modification by plasma treatment was performed on the manufactured microchannel array and the acrylic flat plate. By using a sputtering device (SV, manufactured by ULVAC, INC.), 100 nm of Sio₂ layer was deposited. The contact angle with respect to water was 16°.

[Manufacture of Resin Substrate D]

According to the molded product production process shown in FIGS. 1A to 1H, the resist coating was performed once to form the first resist layer and then exposure and heat-treatment were performed on each layer. Then, the resist coating was performed once to form the second resist layer, and further the resist coating was performed once again to form the third resist layer, and then the exposure and the heat-treatment were performed thereon. A resin microchannel array, as shown in FIG. 10, having a substrate with 16 mm in width, 8 mm in length, and 1.0 mm in thickness on which a micro groove with 7 μm in width and 5 μm in depth and two-step depressions with 40 μm and 80 μm in depth were formed was thereby manufactured.

Surface modification by plasma treatment was performed just like the resin substrate 3. By using a sputtering device (SV, manufactured by ULVAC, INC.), 100 nm of SiO₂ layer was deposited. The contact angle with respect to water was 18°.

[Manufacture of Resin Substrate E]

According to the molded product production process shown in FIGS. 1A to 1H, the resist coating was repeated twice to form the first resist layer and then exposure and heat-treatment were performed on each layer. Further, the resist coating was performed once again to form the second resist layer, and then the exposure and the heat-treatment were performed thereon. A resin microchannel array, as shown in FIG. 8A, having a substrate with 16 mm in width, 8 mm in length, and 1.0 mm in thickness on which a micro groove with 7 μm in width and 5 μm in depth and a depression with 80 μm in depth were formed was thereby manufactured.

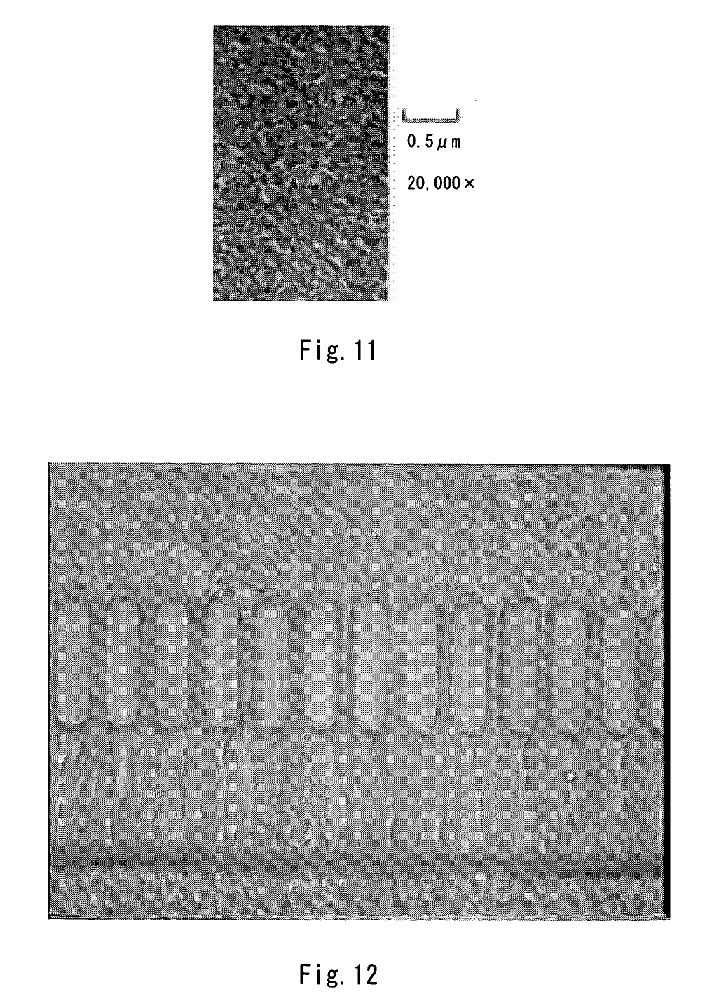

The contact angle with respect to water was 65°. A material used for the injection molding was PARAPET SA, which is acryl manufactured by KURARAY, CO. LTD. The microphase-separated structure of the molded product was observed by TEM. PARAPET SA was composed of a copolymer of two kinds of monomers having different grass transfer temperatures. Due to the variegation with dye, a domain in glass state (black-dyed portion) and a domain in fluid state were microphase-separated at an interval of about 0.1 μm under room temperature (22° C.). FIG. 11 shows a TEM picture.

The number of blood platelets attached was measured. Human blood was in vibration contact with the silicon substrate, the resin molded product 1 and the flat portion 5 for one hour and then cleaned with saline and pure water sequentially. Then, the number of blood platelets attached per 1 cm² in total six positions was checked by using SEM at a magnification of 1000×. The results were 186/cm² in the silicon substrate, 70/cm² in the resin molded product 1, 20/cm² in the resin molded product 5, showing that the microphase-separated structure suppressed the attachment of blood platelets.

### Example 1

Blood Test with Use of Resin Substrate A

After immersing a resin microchannel array into saline in order to prevent the entry of air bubbles, the resin microchannel array was set to a test module. Then, samples were introduced in the order of saline and blood. The blood test checked a passing time of the blood sample from the flow-in through the inlet port at the left end to the flow-out through the outlet port at the right end after flowing through the depression and micro flow channel, and the deformational passing of blood cells by visual observation and the attachment.

The observation with a CCD camera showed that the blood sample had passed through the microchannel and it took 45 seconds for the blood sample of 0.1 ml to pass through the whole channel. The visual observation of the deformational passing of the blood cells showed the flow of the blood cells into the microchannel and the process of partial obstruction due to the attachment of blood platelets or the like, though the entry of air babbles occurred in a part of the depression.

### Example 2

Blood Test with Use of Resin Substrate B

The blood test was successful just like the case of using the resin substrate A. The same blood sample was used. A time required for the blood sample of 0.1 ml to pass through the whole channel was 58 seconds. The use of micro flow channel with a width of 7 μm and a depth of 5 μm, which is smaller than the resin substrate A, resulted in an increase in passing time by about 10 seconds. The visual observation of the deformational passing of the blood cells showed the process that the red blood cell with a diameter of 8 μm passes through the channel as deformed. The observation further showed that the white blood cell with a diameter of 12 μm was immobilized without passing through the microchannel, thus being capable of coping with light, surface plasmon resonance, electrochemical and ultrasonic tests.

Though the entry of air bubbles occurred partly in the depression in the test with the use of the resin substrate A, this test completely eliminated air bubbles by hydrophilization. This was expected to contribute to prevent the attachment of blood cells and also predicted to contribute to reduce a blood passing time. FIG. 12 shows the image in the blood test that was optically photographed by using a CCD camera.

### Example 3

Blood test with Use of Resin Substrate C

The blood test was successful just like the case of using the resin substrate A. The same blood sample was used. A time required for the blood sample of 0.1 ml to pass through the whole channel was 56 seconds. The visual observation of the deformational passing of the blood cells showed the process that the red blood cell with a diameter of 8 μm passed through the channel as deformed just like the resin substrate 2. The observation further showed that the white blood cell with a diameter of 12 μm was immobilized without passing through the microchannel, thus being capable of coping with light, surface plasmon resonance, electrochemical and ultrasonic tests.

Though the entry of air bubbles occurred partly in the depression in the test with the use of the resin substrate A, this test completely eliminated air bubbles by hydrophilization. This was expected to contribute to prevent the attachment of blood cells and also predicted to contribute to reduce a blood passing time.

### Example 4

Blood test with Use of Resin Substrate D

The blood test was successful just like the case of using the resin substrate A. The same blood sample was used. A time required for the blood sample of 0.1 ml to pass through the whole channel was 49 seconds. The visual observation of the deformational passing of the blood cells showed the process that the red blood cell with a diameter of 8 μm passed through the channel as deformed just like the resin substrate 2. The observation further showed that the white blood cell with a diameter of 12 μm was immobilized without passing through the microchannel, thus being capable of coping with light, surface plasmon resonance, electrochemical and ultrasonic tests.

Though the entry of air bubbles occurred partly in the depression in the test with the use of the resin substrate A, this test completely eliminated air bubbles by hydrophilization. This was expected to contribute to prevent the attachment of blood cells and also predicted to contribute to reduce a blood passing time.

Further, the depth of the depression was two-steps of 40 μm and 80 μm to imitate the human capillary, thereby smoothing the flow into the microchannel, which was expected to contribute to reduce a blood passing time in this sample. Making a plurality of steps in the depth of depression was expected to clarify a difference between smooth flow and non-smooth flow of samples in the measurement of a passing time.

### Example 5

Blood test with Use of Resin Substrate E

The blood test was successful just like the case of using the resin substrate A. The same blood sample was used. A time required for the blood sample of 0.1 ml to pass through the whole channel was 66 seconds. The visual observation of the deformational passing of the blood cells showed the process that the red blood cell with a diameter of 8 μm passed through the channel as deformed just like the resin substrate 2. The observation further showed that the white blood cell with a diameter of 12 μm was immobilized without passing through the microchannel, thus being capable of coping with light, surface plasmon resonance, electrochemical and ultrasonic tests.

Although this test showed a longer passing time because of not performing the hydrophilization. However, the use of material having a microphase-separated structure enabled to apply to the blood test.

From the invention thus described, it will be obvious that the embodiments of the invention may be varied in many ways. Such variations are not to be regarded as a departure from the spirit and scope of the invention, and all such modifications as would be obvious to one skilled in the art are intended for inclusion within the scope of the following claims.

What is claimed is:

1. A resin microchannel array comprising:
a first substrate having a plurality of depressions, each depression having an inlet port at one end and an outlet port at another end, and walls sectioning the depressions, each wall having a micro groove connecting the depressions; and
a second substrate having a flat surface bonded or pressure-contacted to a surface of the first substrate,
wherein spaces created by the grooves in a bonded or pressure contacted part between the first substrate and the second substrate serve as flow channels, and
each of a width and a depth of the flow channel is within a range of 1 to 50 μm, and a ratio of the width and the depth of the flow channel is within a range of 1:10 to 10:1.

2. The resin microchannel array according to claim 1, wherein a contact angle of the resin microchannel array with respect to water is from 0.5° to 70°.

3. The resin microchannel array according to claim 1, wherein a place where a blood platelet is attached on a surface of the resin microchannel array is 100 places/cm² and below.

4. The resin microchannel array according to claim 1, wherein each groove has a narrow part with a pitch and depression pattern.

5. The resin microchannel array according to claim 1, wherein each depression has different depths in step-like shape.

6. The resin microchannel array according to claim 1, wherein the resin microchannel array is incinerable as infectious waste.

7. The resin microchannel array according to claim 1, wherein the first substrate and/or the second substrate is transparent.

8. A method of manufacturing a resin microchannel array according to claim 1, comprising:
forming a resist pattern on a substrate;
forming a metal structure by depositing a metal in accordance with the resist pattern formed on the substrate; and
forming a resin microchannel substrate by using the metal structure.

9. A blood test method using a resin microchannel array according to claim 1, the method letting saline, blood sample or reagent flow separately or simultaneously into a single or a plurality of inlet ports of the resin microchannel array and placing a flow control system in a close proximity of an inlet port and/or a close proximity of an outlet port of a test device, thereby repeating an optimal condition for various kinds of blood tests.

10. The blood test method according to claim 9, comprising:
an optical system for applying light to an inlet port and an outlet port of a depression connected through a flow channel or to a flow channel; and
a measurement system for measuring variation in light reflected or transmitted by the flow channel.

11. A blood test method using a resin microchannel array according to claim 1, the method measuring a change in the number of each formed elements of blood at an inlet port and an outlet port of a depression connected through a flow channel or measuring an obstruction state of a groove channel by each formed elements of blood, thereby obtaining flowing characteristics or activity of each formed elements of blood.

12. A blood test method using a resin microchannel array according to claim 1, the method making a difference in concentration of a physiologically active substance between an inlet port and an outlet port of a depression connected through a flow channel so as to cause a white blood cell to move through the flow channel and measuring a change in the number of white blood cell fractions or an obstruction state of a flow channel by the white blood cell, thereby obtaining migration ability and attachment ability of the white blood cell fractions.

13. The blood test method according to claim 9, wherein blood test is performed on a blood sample after exposed to a biologically active substance.

14. The blood test method according to claim 9, wherein blood test is performed by producing fluorescence of each blood cell or fluid element with a fluorescent substance.

15. The blood test method according to claim 9, wherein the method deposits a thin film such as a gold on the first substrate or the second substrate and places a measurement system for detecting a change in permittivity in an inlet port and an outlet port of a depression connected through a flow channel or a flow channel as a change in intensity of reflected light due to surface plasmon resonance.

16. The blood test method according to claim 9, wherein the method places a sensor for electrochemically detecting a slight electric displacement in an inlet port and an outlet port of a depression connected through a flow channel or a flow channel and performs electric amplification for quantitative evaluation.

17. The blood test method according to claim 9, wherein the method places a sensor for ultrasonically detecting a slight frequency change in an inlet port and an outlet port of a depression connected through a flow channel or a flow channel and performs conversion into an electric signal and amplification for quantitative evaluation.
